(19) United States
(12) Patent Application Publication
(10) Pub. No.: US 2011/0208237 A1
(43) Pub. Date: Aug. 25, 2011

(54) FORCEPS FOR PATHOLOGICAL DIAGNOSIS

(75) Inventor: Ying-Chieh Su, Tainan City (TW)

(73) Assignee: CHI MEI MEDICAL CENTER, Yongkang City (TW)

(21) Appl. No.: 12/897,022

(22) Filed: Oct. 4, 2010

(30) Foreign Application Priority Data
Feb. 23, 2010 (TW) 099105152

(51) Int. Cl. A61B 10/06

(52) U.S. Cl. 606/208

(57) ABSTRACT

A forceps for pathological diagnosis includes first and second proximate arm segments including finger-operable grip ends that are movable towards or away from each other, first and second distal arm segments including grasping-jaw ends that are movable towards or away from each other, a coupling member disposed to bring the relative movement between the first and second proximate arm segments into synchronization with that between the first and second distal arm segments, an ultrasonic probe mounted to the grasping-jaw end of the first distal arm segment, and adapted to transmit ultrasonic waves towards a target tissue that is clamped between the grasping-jaw ends and to receive reflected ultrasonic waves traveling from the target tissue, and a shielding member disposed on the grasping-jaw end of the second distal arm segment, and capable of partially reflecting ultrasonic waves transmitting through the target tissue.

### CROSS-REFERENCE TO RELATED APPLICATION

This application claims priority of Taiwanese Application No. 099105152, filed on Feb. 23, 2010.

### BACKGROUND OF THE INVENTION

1. Field of the Invention

The invention relates to a forceps, more particularly to a forceps for pathological diagnosis

2. Description of the Related Art

Cancers, particularly lung cancer, are fatal diseases. Early diagnosis is therefore of extreme importance. Governments, medical centers and medical equipment manufacturers are always looking for new and effective methods and apparatuses for diagnosing and treating cancers.

During diagnosis of pathological changes in the lung, a common procedure is to first locate the pathological change through radiographs or via computerized tomography (CT) scan images, and then perform excision surgery. However, when the area of pathological change is small, it is hard to identify the precise location thereof with the naked eye under videothoracoscopic guide during surgery. In this kind of situation, a surgeon needs to refer to the radiographs or CAT scan images and utilize his/her sense of touch to grope for and feel the area of pathological change. Biopsy has to be performed to evaluate whether the surgeon has correctly located the area of pathological change be fore the actual excision can be performed. A surgeon's clinical experience is heavily relied upon under such circumstance, and it might be time-consuming even for an experienced surgeon. Moreover, there is still the possibility of making a wrong judgment when no other technological assistance can be provided to the surgeon. This greatly increases a patient's risks during surgery, and lengthens operation time.

### SUMMARY OF THE INVENTION

Therefore, the object of the present invention is to provide a forceps that is convenient for pathological diagnosis of organs.

According to the present invention, there is provided a forceps for pathological diagnosis. The forceps is adapted to clamp a target tissue for diagnosis of pathological change in the target tissue, and includes first and second proximate arm segments, first and second distal arm segments, a coupling member, an ultrasonic probe and a shielding member.

Each of the first and second proximate arm segments has a finger-operable grip end and a hinging-side end opposite to each other. The hinging-side ends of the proximate arm segments are hinged to each other such that the finger-operable grip ends of the first and second proximate arm segments are movable towards or away from each other about a first hinge axis.

Each of the first and second distal arm segments has a grasping-jaw end and a hinged-side end opposite to each other. The hinged-side ends of the first and second distal arm segments are hinged to each other such that the grasping-jaw ends of the first and second distal arm segments are movable towards or away from each other about a second hinge axis parallel to the first hinge axis. The grasping-jaw ends are adapted for clamping the target tissue therebetween when moved towards each other about the second hinge axis.

The coupling member is disposed to couple the hinged-side ends with the hinging-side ends so as to bring the relative movement between the first and second proximate arm segments into synchronization with that between the first and second distal arm segments.

The ultrasonic probe is mounted to the grasping-jaw end of the first distal arm segment, and is adapted to transmit ultrasonic waves towards the target tissue and to receive reflected ultrasonic waves traveling from the target tissue.

The shielding member is disposed on the grasping-jaw end of the second distal arm segment, and is capable of partially reflecting ultrasonic waves transmitting through the target tissue.

### BRIEF DESCRIPTION OF THE DRAWINGS

Other features and advantages of the present invention will become apparent in the following detailed description of the preferred embodiments with reference to the accompanying drawings, of which:

FIG. 1 is an exploded schematic view of the first preferred embodiment of a forceps according to the present invention;

FIG. 2 is an assembled schematic view of the first preferred embodiment, illustrating grasping-jaw ends of first and second distal segments being moved away from each other;

FIG. 3 is an assembled schematic view of the first preferred embodiment, illustrating the grasping-jaw ends of the first and second distal segments being moved toward each other so as to clamp a target tissue therebetween;

FIG. 4 is a partially exploded schematic view of the second preferred embodiment of a forceps according to the present invention;

FIG. 5 is an assembled schematic view of the second preferred embodiment; and

FIG. 6 is an assembled schematic view of the third preferred embodiment of a forceps according to the present invention.

### DETAILED DESCRIPTION OF THE PREFERRED EMBODIMENTS

Before the present invention is described in greater detail, it should be noted that like elements are denoted by the same reference numerals throughout the disclosure.

It should be noted herein that the relative proportions and dimensions of the elements shown in the drawings are for illustrative purposes only, and should not be conceived to limit the scope of the present invention.

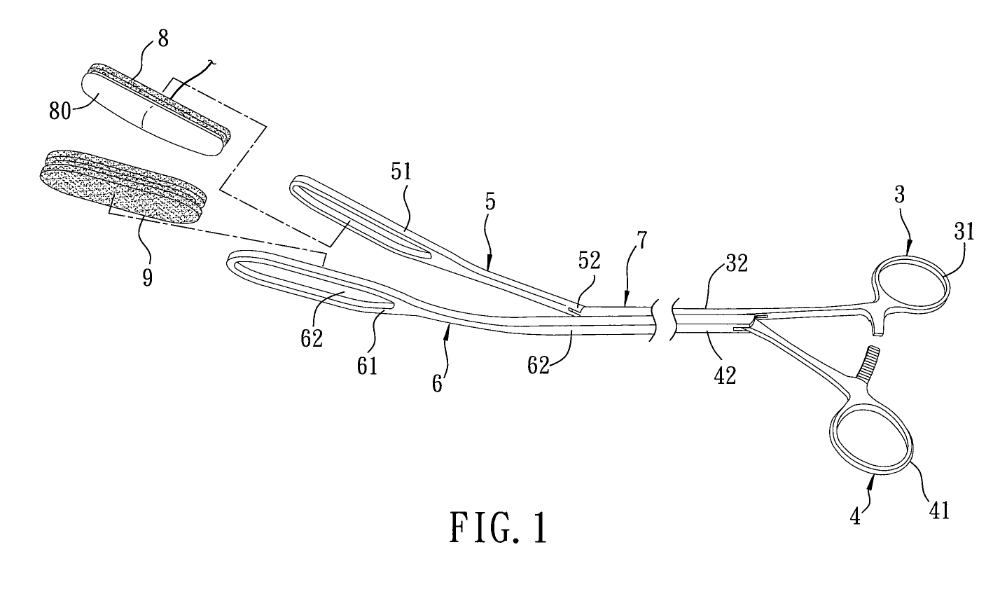

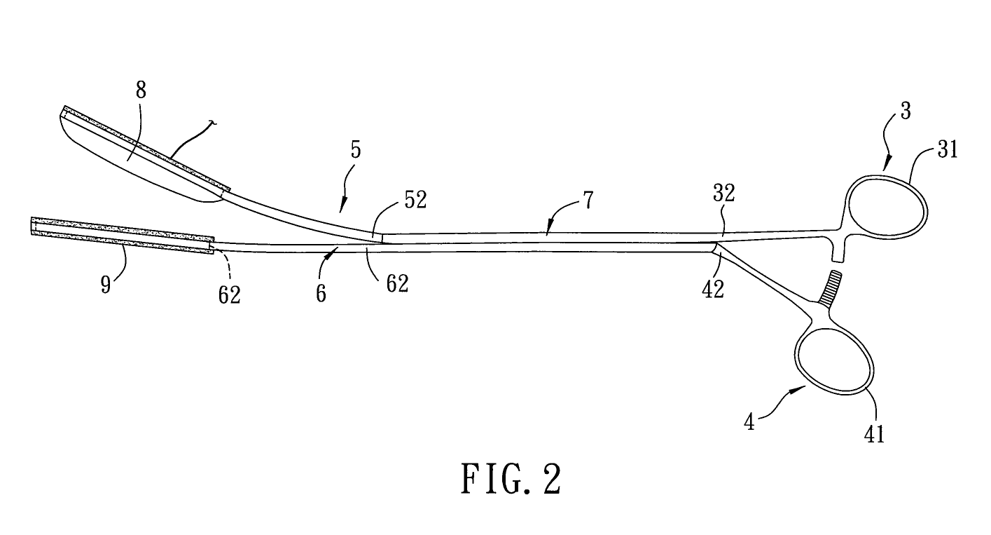

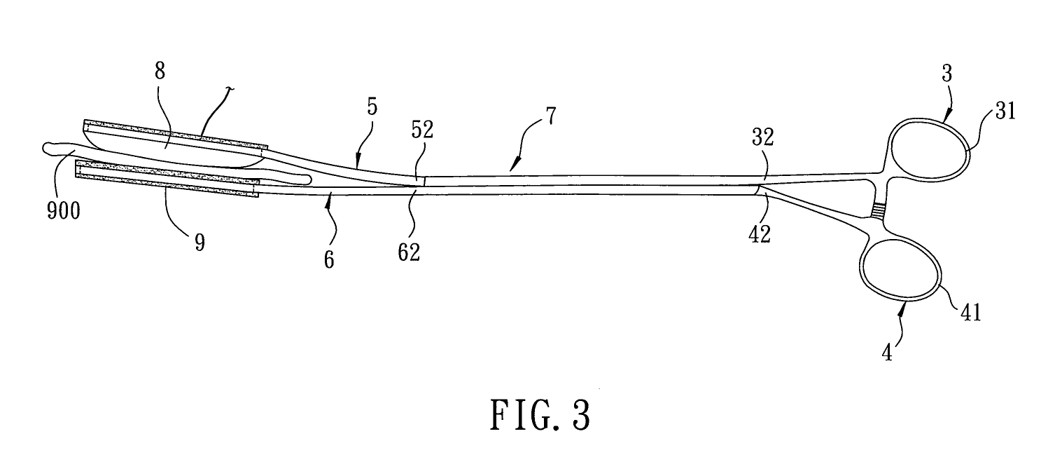

With reference to FIGS. 1, 2 and 3, the first preferred embodiment of a forceps for pathological diagnosis according to the present invention is adapted to clamp a target tissue 900 (only shown in FIG. 3) for diagnosis of pathological change in the target tissue 900, especially during surgical operations where it is necessary to locate the pathological change.

The forceps includes a first proximate arm segment 3, a second proximate arm segment 4, a first distal arm segment 5, a second distal arm segment 6, a coupling member 7, an ultrasonic probe 8, and a shielding member 9. In this embodiment, the forceps is shown to be an elongated forceps particularly used for microsurgery or endoscopic surgery.

Each of the first and second proximate arm segments 3, 4 has a finger-operable grip end 31, 41 and a hinging-side end 32, 42 opposite to each other. The hinging-side ends 32, 42 of the first and second proximate arm segments 3, 4 are hinged to each other such that the finger-operable grip ends 31, 41 of the first and second proximate arm segments 3, 4 are movable towards or away from each other about a first hinge axis.

Each of the first and second distal arm segments 5, 6 has a grasping-jaw end 51, 61 and a hinged-side end 52, 62 opposite to each other. The hinged-side ends 52, 62 of the first and second distal arm segments 5, 6 are hinged to each other such that the grasping-jaw ends 51, 61 of the first and second distal arm segments 5, 6 are movable towards or away from each other about a second hinge axis parallel to the first hinge axis. The grasping-jaw ends 51, 61 are adapted for clamping the target tissue 900 therebetween when moved towards each other about the second hinge axis.

The coupling member 7 is disposed to couple the hinged-side ends 32, 42 of the first and second proximate arm segments 3, 4 with the hinging-side ends 52, 62 of the first and second distal arm segments 5, 6 so as to bring the relative movement between the first and second proximate arm segments 3, 4 into synchronization with that between the first and second distal arm segments 5, 6. In this embodiment, the coupling member 7 includes a coupling mechanism (not shown) that brings the relative movement between the first and second proximate arm segments 3, 4 into synchronization with that between the first and second distal arm segments 5, 6. It should be noted herein that since the feature of this invention does not reside in the translation of movement described above, further details are omitted herein for the sake of brevity.

The ultrasonic probe 8 is mounted to the grasping-jaw end 51 of the first distal arm segment 5, and is adapted to transmit ultrasonic waves towards the target tissue 900 and to receive reflected ultrasonic waves traveling from the target tissue 900. The ultrasonic probe 8 is adapted to be connected to an ultrasonic imaging device (not shown) so as to generate an ultrasonic image with reference to the ultrasonic waves received as a result of the traveling of the reflected ultrasonic waves from the target tissue 900.

The shielding member 9 is disposed on the grasping-jaw end 61 of the second distal arm segment 6, and is capable of partially reflecting ultrasonic waves transmitting through the target tissue 900. The shielding member 9 is made from a biocompatible material, such as silicone gel, whose density permits partial reflection of the ultrasonic waves transmitting through the target tissue 900.

During use, a surgeon may look at a radiograph or a computerized axial tomography (CAT) scan image to get a general picture of where a pathological change might be located, and then use the grasping-jaw ends 51, 61 of the first and second distal arm segments 5, 6 to clamp the target tissue 900 therebetween, such that when the ultrasonic probe 8 is activated (by an ultrasonic equipment (not shown)) (i.e., ultrasonic waves are transmitted toward the target tissue 900), ultrasonic waves received by the ultrasonic probe 8 as a result of the traveling of the reflected ultrasonic waves from the target tissue 900 can be used to form an ultrasonic image by the ultrasonic imaging device (not shown) for reference by the surgeon to diagnose where the pathological change is located. Since the amount of ultrasonic waves reflected from the target tissue 900 differs as the density of the target tissue 900 varies, and since the density of a pathologically-changed portion of an organ is significantly different from that of a normal portion of the same organ, the ultrasonic image of the target tissue 900 clamped by the grasping-jaw ends 51, 61 of the first and second distal arm segments 5, 6 can be utilized for diagnosis. The surgeon can then perform excision surgery on the correct portion of the organ.

The ultrasonic probe 8 and the shielding member 9 are to be cleansed and sterilized for future use.

In this embodiment, the grasping-jaw end 61 of the second distal arm segment 6 is formed with a mounting hole 62, and the shielding member 9 is configured to be fitted in the mounting hole 62. The mounting hole 62 is formed because without it, an enormous amount of ultrasonic signals will reflect off the face of the grasping-jaw end 61 (which is made of metal), causing a bright spot in the resulting ultrasonic image that obstruct diagnosis. The shielding member 9 is provided to permit thereat partial reflection of the ultrasonic waves transmitting through the target tissue 900 so as to ensure that the resulting ultrasonic image will not be insufficiently clear for proper diagnosis.

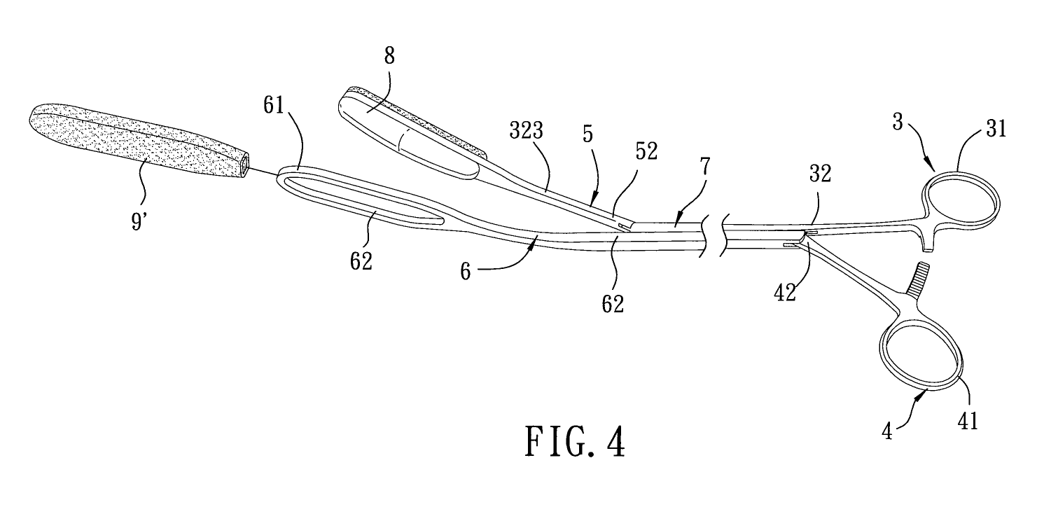

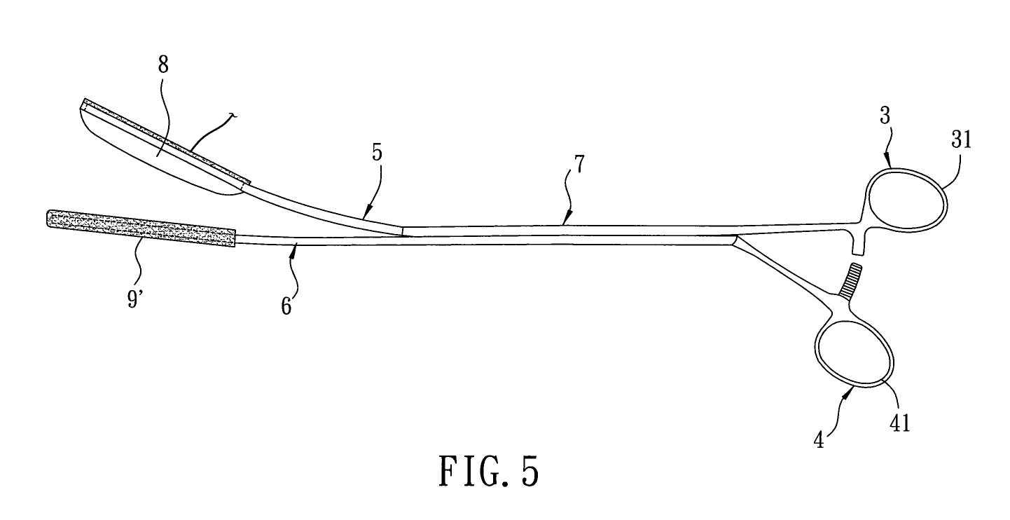

With reference to FIG. 4 and FIG. 5, the second preferred embodiment of a forceps according to the present invention differs from the first preferred embodiment in that the shielding member 9′ of the second preferred embodiment is in the form of a covering that covers the grasping-jaw end 61 of the second distal arm segment 6.

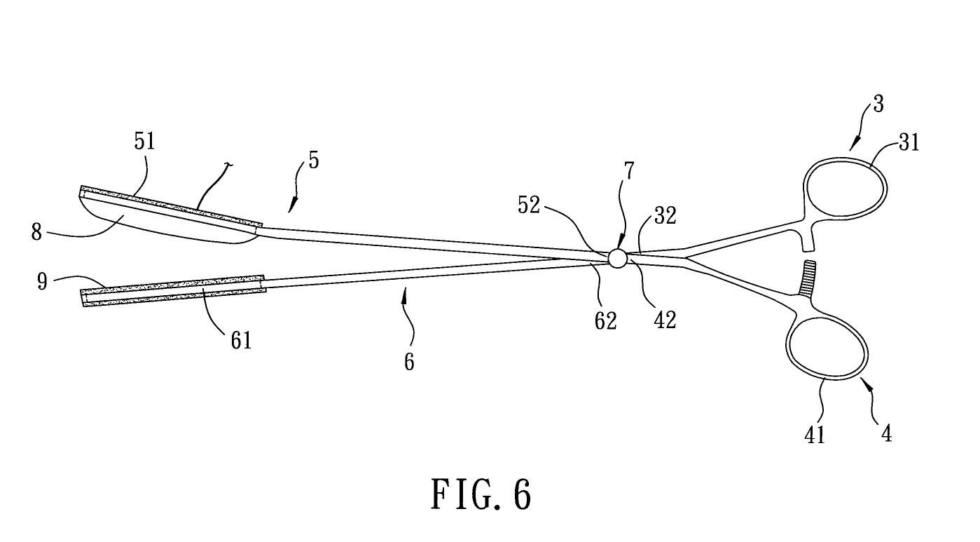

With reference to FIG. 6, the third preferred embodiment of a forceps according to the present invention differs from the previous embodiments in that the coupling member 7 is configured so as to bring the first axis in alignment with the second hinge axis. In addition, the hinging-side end 32, 42 of each of the first and second proximate arm segments 3, 4 is integrally formed with the hinged-side end 62, 52 of a corresponding one of the first and second distal arm segments 6, 5. In other words, the third embodiment operates in a way similar to a pair of scissors.

In summary, through the structural configuration of the grasping-jaw ends 51, 61 of the first and second distal arm segments 5, 6, and the provision of the ultrasonic probe 8 and the shielding member 9, the forceps of the present invention is adapted to clamp a target tissue 900, and to facilitate diagnosis of pathological change in the target tissue 900 by a surgeon so as to assist the surgeon to quickly locate the areas of the pathological change, and to reduce the time of surgery to thereby reduce a patient's risks during the surgery.

While the present invention has been described in connection with what are considered the most practical and preferred embodiments, it is understood that this invention is not limited to the disclosed embodiments but is intended to cover various arrangements included within the spirit and scope of the broadest interpretation so as to encompass all such modifications and equivalent arrangements.

What is claimed is:

1. A forceps for pathological diagnosis adapted to clamp a target tissue for diagnosis of pathological change in the target tissue, said forceps comprising:
first and second proximate arm segments each having a finger-operable grip end and a hinging-side end opposite to each other, said hinging-side ends of said first and second proximate arm segments being hinged to each other such that said finger-operable grip ends of said first and second proximate arm segments are movable towards or away from each other about a first hinge axis;
first and second distal arm segments each having a grasping-jaw end and a hinged-side end opposite to each other, said hinged-side ends of said first and second distal arm segments being hinged to each other such that said grasping-jaw ends of said first and second distal arm segments are movable towards or away from each other about a second hinge axis parallel to the first hinge axis, said grasping-jaw ends being adapted for clamping the target tissue therebetween when moved towards each other about the second hinge axis;
a coupling member disposed to couple said hinged-side ends with said hinging-side ends so as to bring the relative movement between said first and second proximate arm segments into synchronization with that between said first and second distal arm segments;
an ultrasonic probe mounted to said grasping-jaw end of said first distal arm segment, and adapted to transmit ultrasonic waves towards the target tissue and to receive reflected ultrasonic waves traveling from the target tissue; and
a shielding member disposed on said grasping-jaw end of said second distal arm segment, and capable of partially reflecting ultrasonic waves transmitting through the target tissue.

2. The forceps as claimed in claim 1, wherein said grasping-jaw end of said second distal arm segment is formed with a mounting hole, and said shielding member is configured to be fitted in said mounting hole.

3. The forceps as claimed in claim 1, wherein said grasping-jaw end of said second distal arm segment is formed with a mounting hole, and said shielding member is in the form of a covering that covers said grasping-jaw end of said second distal arm segment.

4. The forceps as claimed in claim 1, wherein said ultrasonic probe is adapted to be connected to an ultrasonic imaging device so as to generate an ultrasonic image with reference to the ultrasonic waves received as a result of the traveling of the reflected ultrasonic waves from the target tissue.

5. The forceps as claimed in claim 1, wherein said shielding member is made from a biocompatible material.

6. The forceps as claimed in claim 1, wherein said coupling member is configured so as to bring said first axis in alignment with said second hinge axis.

7. The forceps as claimed in claim 1, wherein said hinging-side end of each of said first and second proximate arm segments is integrally formed with said hinged-side end of a corresponding one of said first and second distal arm segments.
